SEQUENCE LISTING (9 sequences)

SEQ ID NO: 1 - protein, 22 aa, Unknown
  PEPTIDE: IL-8 peptide antagonist
TWFEDEFANATGMPPVEKDYSP

SEQ ID NO: 2 - protein, 22 aa, Unknown
  PEPTIDE: IL-8 peptide antagonist
TWFEDEHANATGMPPVEKDYSP

SEQ ID NO: 3 - protein, 22 aa, Unknown
  PEPTIDE: IL-8 peptide antagonist
TWFEDEFHNATGMPPVEKDYSP

SEQ ID NO: 4 - protein, 22 aa, Unknown
  PEPTIDE: IL-8 peptide antagonist
TWFEDEFAHATGMPPVEKDYSP

SEQ ID NO: 5 - protein, 22 aa, Unknown
  PEPTIDE: IL-8 peptide antagonist
TWFEDEFANHTGMPPVEKDYSP

SEQ ID NO: 6 - protein, 22 aa, Unknown
  PEPTIDE: IL-8 peptide antagonist
TWFEDEFANAHGMPPVEKDYSP

SEQ ID NO: 7 - protein, 22 aa, Unknown
  PEPTIDE: IL-8 peptide antagonist
TWFEDEFANATHMPPVEKDYSP

SEQ ID NO: 8 - protein, 22 aa, Unknown
  PEPTIDE: IL-8 peptide antagonist
TWFEDEFANATGHPPVEKDYSP

SEQ ID NO: 9 - protein, 22 aa, Unknown
  PEPTIDE: IL-8 peptide antagonist
TWFEDEFANATGMPPVEKDYSP

(19) United States
(12) Patent Application Publication
(10) Pub. No.: US 2004/0152634 A1
(43) Pub. Date: Aug. 5, 2004

(54) Chemokine antagonists and uses thereof

(76) Inventor: Javier V. Navarro, Houston, TX (US)

Correspondence Address: Benjamin Aaron Adler, ADLER & ASSOCIATES, 8011 Candle Lane, Houston, TX, 77071, US

(21) Appl. No.: 10/744,876

(22) Filed: Dec. 23, 2003

Related U.S. Application Data
(60) Provisional application No. 60/436,253, filed on Dec. 24, 2002.

(51) Int. Cl. A61K 38/17; C07K 14/715

(52) U.S. Cl. 514/12; 530/350

(57) ABSTRACT

The present invention provides a chemokine peptide antagonist comprising the N-terminal domain of a CXCR1 receptor. The native amino acid(s) of such N-terminal domain is replaced by one or more histidine residues, thereby forming Zn(II) binding site(s) in said N-terminal domain. The antagonist specifically binds a chemokine in the presence of Zn(II), thereby neutralizing the action of the chemokine. Representative examples of chemokine peptide antagonist effective against IL-8 are disclosed herein. Also provided are pharmaceutical compositions containing the chemokine peptide antagonists of the present invention and methods of using such pharmaceutical compositions.

### CROSS-REFERENCE TO RELATED APPLICATION

This non-provisional application claims benefit of provisional U.S. Serial No. 60/436,253, filed Dec. 24, 2002, now abandoned.

### FEDERAL FUNDING LEGEND

This invention was supported in part by National Institutes of Health grant RO1-AI34031. Consequently, the federal government has certain rights in this invention.

### BACKGROUND OF THE INVENTION

1. Field of the Invention

The present invention relates generally to the fields of chemokine biology and pharmacology. More specifically, it relates to novel chemokine antagonists and uses thereof to treat chemokine related diseases.

2. Description of the Related Art

Chemokines are potent chemoattractant cytokines that trigger cell-specific migration of leukocytes from the circulation to sites of inflammation. Chemokines also play key roles in regulating cell trafficking during brain development, angiogenesis, neoplastic growth, myo-fibroblast activation, and viral infections. The majority of chemokines are secretory proteins produced by many cell types after induction, and exert their effects locally in paracrine or autocrine fashion. Chemokine receptors belong to the superfamily of the heptahelical G protein-coupled receptor (GPCR) membrane proteins, which mediate a wide variety of biological processes, including neurotransmission and hormonal control of virtually all physiological responses to perception of taste, smell, light and pain.

Chemokines are classified according to the location and number of cysteine residues in the N-terminus, and are divided into four major types: CXCL, containing two cysteines separated by a single residue; CC, consisting of two adjacent cysteines; XCL, containing a lone cysteine; and CX3C, containing two cysteines separated by three residues. Analogously, chemokine receptors are classified as CXCR, CCR, XCR, and CX3CR, according to their activation by chemokine subtypes (Baggiolini, 2001).

Generally, chemokines are water-soluble proteins of 70-120 amino acids in length. Structural analysis of chemokines by X-ray crystallography and NMR spectroscopy revealed a common structural fold, despite the low degree of sequence homology and/or primary function. Their polypeptide chains are folded into three antiparallel β-strands onto which is packed a C-terminal α-helix (FIG. 1).

Chemokines: Targets for Anti-Inflammatory Therapy

Chemokines and their receptors have attracted considerable interest as novel targets for inflammatory diseases, as well as containment of HIV-1. Inflammatory disorders such as arthritis are tissue specific, characterized by accumulation of a subset of leukocytes at the site of inflammation. Interestingly, chemokines trigger and regulate leukocyte trafficking to the site of inflammation in a tissue-specific fashion as chemokine receptors label specific leukocytes.

Neutralization of the chemokine system as a conceptual mechanism of tissue-specific anti-inflammatory therapy is in contradistinction to current anti-inflammatory therapies such as steroids, which are potent and broad immunosuppressors. Anti-chemokine antibodies have been employed to test the role of chemokines in inflammation. Neutralization of chemokines has been shown to be efficient anti-inflammatory therapies, better than the most potent immunosuppressive drugs such as FK506.

It is highly desirable, however, to develop small compounds against the chemokine receptor system, as they are preferred by pharmaceutical companies over monoclonal antibodies. At least two targets for controlling inflammation in a leukocyte-specific fashion can be identified: one is the inhibition of the chemokine receptor and the other is neutralization of its chemokine. Structure-based design of small compounds inhibiting chemokine receptors selectively has been precluded due to difficulties in solving the structure of G protein-coupled receptors. To date, the atomic structure of just one heptahelical G protein-coupled receptor, rhodopsin, has been solved. On the other hand, the structure of chemokines have been solved by NMR and crystallography, which could assist in developing structure-based strategies to design novel anti-chemokines using small molecules (Baldwin et al., 1991).

The chemokine CXCL8 (also known as interleukin 8, IL-8) and its cognate receptor CXCR1 are the best characterized, and are regarded as the paradigm of the chemokine receptor systems. IL-8 is secreted by many cell types in response to inflammatory stimulus or injury, and induces chemotaxis and activation of neutrophils. Furthermore, IL-8 binds to several chemokine receptors including CXCR1, CXCR2, Duffy antigen, and the virus-derived receptors KSHV heptahelical G protein-coupled receptor and ECRF3.

Neutrophils are the most abundant inflammatory cell type in the joints of patients with rheumatoid arthritis (RA), and IL-8 is elevated in the synovial fluid of rheumatoid arthritis patients. Macrophages from rheumatoid arthritis synovial tissue secrete IL-8 constitutively, in contrast to macrophages from normal patients. In addition, IL-8 is an important contributor to the angiogenic activity found in the inflamed rheumatoid arthritis joint. Rheumatoid arthritis patients treated with a high dose of methylprednisolone displayed a significant decrease in IL-8 expression in synovial tissue biopsies, together with an excellent clinical response.

These observations are in good agreement with results in animal models of crystal-induced arthritis, where IL-8 was the single factor determining the development of clinical signs and symptoms of arthritis. Most importantly, neutralization of IL-8 attenuated crystal-induced arthritis in an animal model. These findings agree with the view that the recruitment of neutrophils towards the synovial compartment is mediated by IL-8. Furthermore, neutrophils at the site of inflammation are activated and release collagenase, elastase, gelatinase, myeloperoxidase, prostaglandins, and leukotrienes. Activated neutrophils will also release proteins such as fibronectin and cytokines including IL-1β, TNF-α, and IL-8. The increased recruitment of neutrophils into the synovial compartment and their prolonged lifespan in response to IL-8 are very important in the development of clinical symptoms, including swelling and pain. Therefore, neutralization of IL-8 could be an effective anti-rheumatic treatment.

Historically, most anti-inflammatory drugs have targeted mechanisms that were too fundamental, thereby rendering the drugs either broadly immunosuppressive (e.g. steroids, cyclosporine) or capable of doing little other than to control disease symptoms (antihistamines, COX-2 inhibitors). So far the search for the wonder drug that balances efficiency and selectivity has been unsuccessful. The discovery of chemokines and their receptors has opened new avenues for anti-inflammatory therapy, giving hope for replacing or complementing the imprecise and imperfect anti-inflammatory drugs in use today. The fact that chemokine receptors are expressed in a tissue-specific fashion argues that targeting the chemokine receptor system will provide a means of treating inflammatory disorders in a tissue-specific fashion. For example, a drug that inhibits an inflammatory response in the skin for a disease like psoriasis should spare the mucosal immune system.

The first gene encoding the IL-8 receptor was isolated in 1991. CXCR1 showed tissue-specific expression —CXCR1 is expressed almost exclusively in neutrophils. Therefore, CXCR1 and its homologous CXCR2, have been regarded as drug targets for several inflammatory disorders. The potential sites for therapeutic intervention in the chemokine system, including upstream and downstream components are illustrated in FIG. 2.

The current targets for developing anti-inflammatory therapeutics are the chemokines and their cognate receptors (FIG. 3). Two strategies have been applied to design anti-chemokine or inhibitors of chemokine receptors. One is the generation of neutralizing monoclonal antibodies against the chemokines or their receptors, and the other is the discovery of small molecules that block the chemokine receptor. Several small molecules have been discovered to block the function of chemokine receptors by binding to sites within the transmembrane domains.

Both approaches are routinely carried out in many laboratories; however, there are two major problems that need to be addressed. First of all, since the small molecule inhibitors are binding to the conserved transmembrane domains of the receptors, these molecules most likely are not specific, because the binding selectivity of chemokines is determined by the highly variable extracellular domains of the chemokine receptors. Indeed, piperidine-containing compounds block several chemokine receptors as well as other heptahelical G protein-coupled receptors including receptors for somatostatin, C5a, tackynin, neuropeptide Y and cholecystokinin. Secondly, although monoclonal antibody therapies are highly specific for neutralizing the chemokines or the receptors, antibodies are expensive and cumbersome to produce, are not very stable upon long storage, can be immunogenic, are difficult to administer, and are not the inhibitors preferred by the pharmaceutical industry.

The prior art is thus deficient in providing effective chemokine antagonists that target the chemokine instead of the chemokine receptors. The present invention fulfills this long-standing need and desire in the art by describing novel chemokine antagonists and uses thereof.

### SUMMARY OF THE INVENTION

This invention involves the design of agents based on the molecular interactions between interleukin-8 (IL-8) and its cognate chemokine receptor (CXCR1). Since IL-8 mediates the migration of neutrophils to sites of inflammation and tissue injury, neutralizing IL-8 with peptides derived from CXCR1 is a novel approach to the development of potent agents such as anti-inflammatory compounds. Indeed, a peptide of 21-amino acids in length, derived from the human CXCR1, binds with low affinity to human IL-8, thus neutralizing the action of IL-8. Novel peptides that bind IL-8 with high affinity and block the action of IL-8 were specifically designed. These peptides were derived from the rabbit or human CXCR1, which were further modified by replacing native residues with histidine residues (His-peptides). These His-peptides bind to IL-8 with high affinity in the presence of Zn (II), thus blocking the action of IL-8. These novel peptides can be used to treat inflammatory disorders and tissue injuries mediated by IL-8, such as ischemia-reperfusion injury, psoriasis, and microbial infections. Furthermore, the same strategies can be applied to develop novel chemokine antagonists which can be used for preventing chemokine related pathologies including HIV-1 infection, allergies, arthritis, and arteriosclerosis.

The instant invention provides a chemokine peptide antagonist, a pharmaceutical composition comprising this antagonist, and method of using this pharmaceutical composition. In one embodiment of the present invention, the chemokine peptide antagonist comprises the N-terminal domain of a CXCR1 receptor, wherein native amino acid(s) of the domain is replaced by one or more histidine residues, thereby forming in the domain Zn(II) binding site(s) that binds the chemokine in the presence of Zn(II).

### BRIEF DESCRIPTION OF THE DRAWINGS

FIG. 1 shows a ribbon representation of CXCL8 or IL-8 as determined by X-ray crystallography and NMR spectroscopy.

FIG. 2 shows potential therapeutic targets in the chemokine signaling pathway.

FIG. 3 shows chemokines and their receptors as current therapeutic targets for anti-inflammation.

FIG. 4 shows the IL-8 ribbon representation is superimposed on the IL-8 surface representation bound to the N-terminal peptide of CXCR1 in sticks. The orientation of His18 of IL-8 is in orange. The figure was created using the accession numbers 1ILP and 1ILQ and the program PyMol.

FIG. 5 shows the same representation as in FIG. 4 except that a Zn(II) coordination site was modeled at the interface by replacing Met12 by His (show in blue) in the N-terminal peptide of CXCR1. His 18 of IL-8 is colored in orange.

FIG. 6 shows the inhibition of the ¹²⁵I-IL-8 binding to neutrophil membranes by a synthetic N-terminal peptide CXCR1 (21-residues in length in which Met 12 was replaced by a His to create a Zn(II) binding site) in the presence and absence of 100 μM ZnCl₂.

FIG. 7 shows the effects of IL-8 mutants on β-glucuronidasesecretion. Human neutrophils were treated with several concentrations of IL-8 or IL-8 mutants, and degranulation of β-glucuronidase was assayed as described Bary et al. IL-8 ( [symbol] ), H18A ( [symbol] ), H18D ( [symbol] ), F21A ( [symbol] ), F21D ( [symbol] ), H18A/F21A ( [symbol] ), H18A/F21D ( [symbol] ), H18A/F21S (▪), and H18D/F21D ( [symbol] ) were added at the indicated concentrations. The percentages of β-glucuronidase release in the presence of 1 μM IL-8 is referred as 100%. Values are means of triplicate determinations, and the bars of each point represent the standard errors.

FIG. 8 shows that the IL-8 mutants are poor activators of superoxide production. Time course of IL-8 or IL-8 mutant-induced superoxide production, assayed by monitoring the fluorescence intensity of stable fluorescent product 2,2′-dihydroxybiphenyl-5,5′-acetate as described (Wymann et al., 1987). Panel A: IL-8 (1), H18A (2), F21A (3), and H18A/F21A (4) were added to neutrophils at a final concentration of 100 nM. Panel B:IL-8 (1), H18D (2), F21D (3), F21S (4), H18A/F21D (5), H18A/F21S (6), and H18D/F21D (7) were added to neutrophils at a final concentration of 100 nM. Arrows indicate the addition of IL-8 or the IL-8 mutant. The horizontal bar is the 1-min interval. The vertical bar corresponds to the change of fluorescence elicited by 50 nM H₂O₂. These are representative records of three independent determinations.

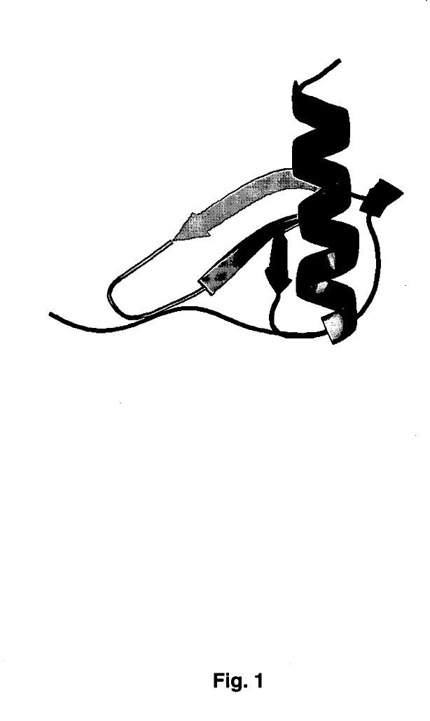

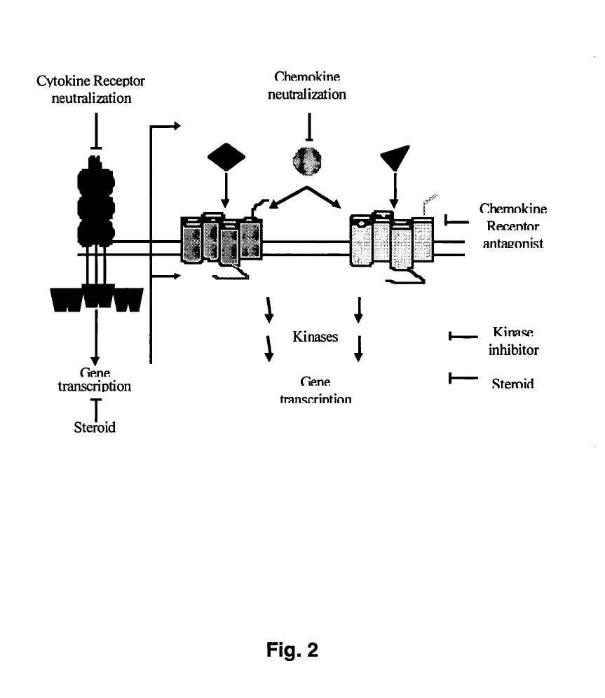

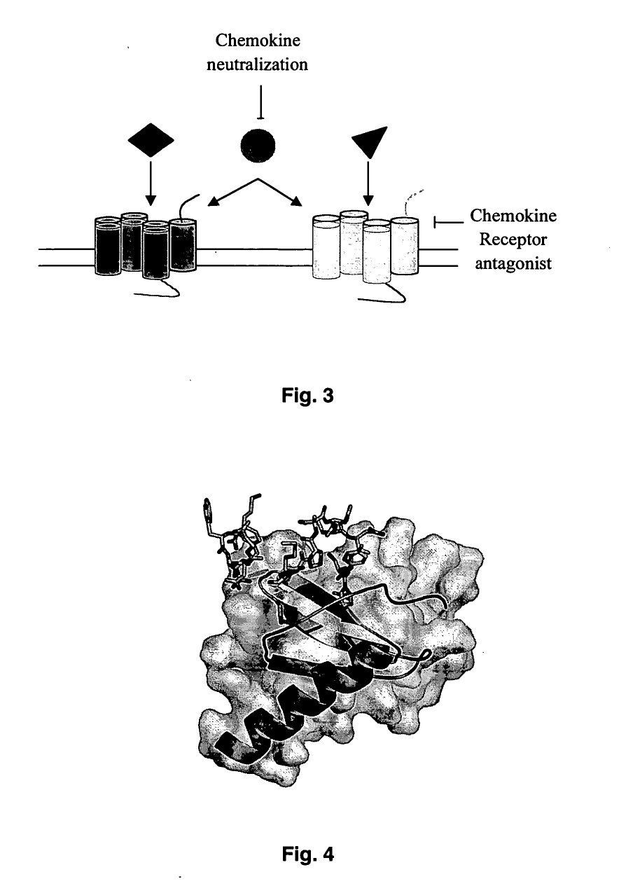

Other and further aspects, features, and advantages of the present invention will be apparent from the following description of the presently preferred embodiments of the invention. These embodiments are given for the purpose of disclosure.

### DETAILED DESCRIPTION OF THE INVENTION

The present invention discloses the design of novel agents based on the atomic structure of IL-8 bound to the N-terminal domain of CXCR1. This strategy is based on the concept that this domain of CXCR1 confers high affinity and selectivity to bind IL-8. Using the NMR structure of the IL-8 bound to a synthetic N-terminal fragment from human CXCR1, peptides which bind IL-8 and neutralize its action were designed and synthesized. Several peptides derived from the N-terminal domain of CXCR1 have been previously synthesized and shown to be specific in neutralizing IL-8; however, they bind IL-8 with very low affinity (Skelton et al., 1999).

The novel peptides disclosed herein display high-affinity binding to IL-8 as well as neutralization of IL-8 action. The neutralizing peptides are relatively small (MW in the 2,000 daltons range) and water-soluble. The neutralizing peptides are as specific as the monoclonal antibodies, as both bind to the same site in CXCR1 (Suetomi et al., 1999). These anti-IL-8 peptides are not immunogenic, as they are derived from the naturally expressed CXCR1.

There are many advantages of using small peptides directed against IL-8 to control inflammation. Since the key residues involved in the selectivity are known, one can design peptides with high binding affinity to IL-8 by residue substitutions and molecular modeling, thus limiting the large screening assays. The neutralizing peptides are readily synthesized by conventional procedures such as the Fmoc-based solid phase synthesis.

The present invention discloses 21-amino acids peptide as it has been shown that this is the minimum size of the N-terminus of CXCR1 that can effectively block the binding of IL-8 to its cognate receptor. The potency of the anti-IL-8 peptides can be enhanced by creating Zn(II) coordination sites through the introduction of 2 or 3 histidine (His) in the peptides. The positions of the histidine can be chosen on the basis of the results of single His-substitutions in the peptide (His scanning). Previous studies have shown that increasing the number of Zn(II) coordination sites at the interface of protein-protein complex enhances the binding affinity by several order of magnitude. Moreover, the concentration of Zn(II) used in the binding assays disclosed herein is 100 μM, which is in the range of the plasma levels. By increasing the number of Zn(II) coordination sites, lower concentration of Zn(II) can be used. This is important for in vivo uses because the amount of free Zn in vivo is limiting.

In a certain embodiment, the present invention is directed to a chemokine peptide antagonist comprising the N-terminal domain of a CXCR1 receptor. The native amino acid(s) of this domain is replaced by one or more histidine residues, thereby forming in said N-terminal domain Zn(II) binding site(s) that binds a chemokine in the presence of Zn(II). In one aspect, the antagonist specifically binds the chemokine in the presence of Zn(II), thus neutralizing the action of the chemokine. Generally, the chemokine peptide antagonist acts as a decoy preventing the interaction of the chemokine with its cognate receptor. Representative examples of chemokine peptide antagonist effective against IL-8 are selected from the group consisting of SEQ ID NOs:1-9.

The present invention is also directed to a pharmaceutical composition comprising the chemokine peptide antagonist of the present invention, and a method of using such pharmaceutical composition to treat an individual having or at risk of having a disorder or disease mediated at least in part by chemokines. In a preferred aspect, the chemokine is IL-8 and the composition comprises a peptide selected from the group consisting of SEQ ID NOs:1-9.

Generally, the pharmaceutical compositions of the present invention may be used to treat any disorder or disease mediated by chemokines. Representative examples of chemokine mediated diseases or disorders include ischemia-reperfusion injury, psoriasis, microbial infections, HIV-1 infection, allergies, arthritis, arteriosclerosis, inflammation, chronic and acute inflammation, gout, acute pseudogout, acute gouty arthritis, rheumatoid arthritis, osteoarthritis, allograft rejection, chronic transplant rejection, asthma, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, septic shock, endotoxic shock, urosepsis, glomerulonephritis, lupus nephritis, thrombosis, graft vs. host reaction, angiogenesis, NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

This invention provides pharmaceutical compositions containing chemokine peptide antagonists of the present invention. In one embodiment, such compositions include chemokine peptide antagonists of the invention in an effective amount, meaning a therapeutically or prophylactically effective amount, sufficient to modulate CXCR-1 and CXCR-2 activity, and a pharmaceutically acceptable carrier. In other embodiments, the compositions of the invention may include chemokine peptide antagonists of the invention in a therapeutically or prophylactically effective amount sufficient to modulate the activity of IL-8, and a pharmaceutically acceptable carrier. Chemokine peptide antagonists of the invention may also be used in combination with other compositions and procedures for the treatment of diseases.

A “therapeutically effective amount” refers to an amount effective, at dosages and for periods of time necessary, to achieve the desired therapeutic result, such as modulation of CXCR-1, CXCR-2 or IL-8 activity. A therapeutically effective amount of a chemokine peptide antagonist of the present invention may vary according to factors such as the disease state, age, sex, and weight of the individual, and the ability of chemokine peptide antagonists of the present invention to elicit a desired response in the individual. Dosage regimens may be adjusted to provide the optimum therapeutic response. A therapeutically effective amount is also one in which any toxic or detrimental effects of chemokine peptide antagonists of the present invention are outweighed by the therapeutically beneficial effects.

A “prophylactically effective amount” refers to an amount effective, at dosages and for periods of time necessary, to achieve the desired prophylactic result, such as modulation of CXCR-1, CXCR-2 or IL-8 activity. A prophylactically effective amount can be determined as described above for the therapeutically effective amount. Typically, since a prophylactic dose is used in subjects prior to or at an earlier stage of disease. The prophylactically effective amount will be less than the therapeutically effective amount.

In particular embodiments, a preferred range for therapeutically or prophylactically effective amounts of chemokine peptide antagonists may be 0.1 nM-0.1 M, 0.1 nM-0.0SM, 0.05 nM-15 μM or 0.01 nM-10 μM. Alternatively, total daily dose may range from about 0.001 to about 100 mg/kg, or up to 10 mg/kg or up to 1 mg/kg of patients body mass. Dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the methods of the invention.

Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages; each unit containing a predetermined quantity of active chemokine peptide antagonist calculated to produce the desired therapeutic effect in association with the required pharmaceutical carrier. The specification for the dosage unit forms are dictated by and directly dependent on (a) the unique characteristics of the active chemokine peptide antagonist and the particular therapeutic effect to be achieved, and (b) the limitations inherent in the art of compounding such an active chemokine peptide antagonist for the treatment of sensitivity in individuals.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active chemokine peptide antagonist, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, compounds of the present invention can be administered in a time release formulation, for example in a composition which includes a slow release polymer. The active chemokine peptide antagonists can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating chemokine peptide antagonists of the present invention in the required amount in an appropriate solvent with one or a combination of ingredients enumerated above, followed by filtered sterilization. Generally, dispersions are prepared by incorporating the active compound into a sterile vehicle which contains a basic dispersion medium and other required ingredients enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution. In accordance with an alternative aspect of the invention, chemokine peptide antagonists of the invention may be formulated with one or more additional chemokine peptide antagonists that enhance the solubility of compounds of the invention.

Pharmaceutically acceptable salts include salts that are well known to those skilled in the art such as basic salts of inorganic and organic acids, such as hydrochloric acid, hydrobromic acid, sulphuric acid, phosphoric acid, methane sulphonic acid, ethane sulphonic acid, acetic acid, malic acid, tartaric acid, citric acid, lactic acid, oxalic acid, succinic acid, fumaric acid, maleic acid, benzoic acid, alicylic acid, phenylacetic acid and mandelic acid. In alternative embodiments, pharmaceutically acceptable cation salts may include alkaline, alkaline earth, ammonium and quaternary ammonium cations.

In accordance with another aspect of the invention, therapeutic compositions of the present invention, comprising a chemokine peptide antagonist of the invention, may be provided in containers having labels that provide instructions for use of chemokine peptide antagonists to treat chemokine or chemokine receptor mediated diseases. In some embodiments, such diseases may include inflammation, acute inflammation, chronic inflammation, psoriasis, gout, acute pseudogout, acute gouty arthritis, arthritis, rheumatoid arthritis, osteoarthritis, allograft rejection, chronic transplant rejection, asthma, mononuclear-phagocyte dependent lung injury, idiopathic pulmonary fibrosis, sarcoidosis, focal ischemia, atopic dermatitis, chronic obstructive pulmonary disease, adult respiratory distress syndrome, acute chest syndrome in sickle cell disease, inflammatory bowel disease. Crohn's disease, ulcerative colitis, septic shock, endotoxic shock, urosepsis, glomerulonephritis, thrombosis, graft vs. host reaction, angiogenesis, NSCLC, human ovarian cancer, and human pancreatic adenocarcinoma.

An alternative aspect of the invention, chemokine or chemokine receptor mediated diseases may include cancers susceptible to anti-angiogenic treatment, including both primary and metastatic solid tumors, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas).

In some aspects of the invention, compounds of the invention may also be useful in treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, compounds of the invention may be useful in the prevention of metastases from the tumors described above either when used alone or in combination with radiotherapy and/or other chemotherapeutic agents.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Design of High-Affinity Anti-IL-8 Peptides Based On the Atomic Structure of Il-8 Bound to the N-Terminal Fragment of CXCR1

Ideally, structure-based design of IL-8 antagonists requires identification of the orientation and proximity of key side chains at the interface of the IL-8/CXCR1 complex. Unfortunately, the atomic structure of CXCR1 bound to ligands, like any G protein-coupled receptor except rhodopsin, is unknown and represents a major challenge in structural biology. Fortunately, a peptide corresponding to the N-terminal domain of CXCR1 binds IL-8 and neutralizes the binding of IL-8 to the native CXCR1 receptor (Gayle et al., 1993). The NMR solution structure of IL-8 bound to the CXCR1-based peptide revealed that a cleft between a loop and a β hairpin constitutes part of the receptor site on IL-8 (Skelton et al., 1999). Nine residues (Pro21-Pro29) of CXCR1 bind to the cleft in an extended fashion via hydrophobic interactions (FIG. 4).

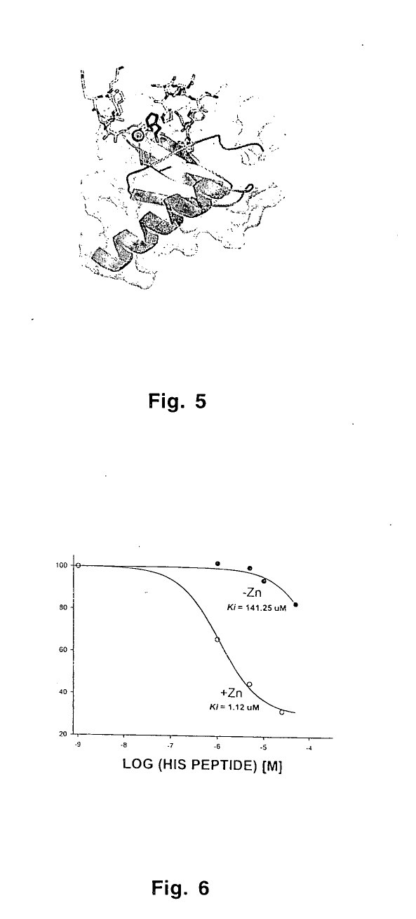

This structural work laid the foundation for the structure-based design of chemokine antagonists. As previously indicated, however, the small peptide binds with low affinity to IL-8 (˜100 μM). To increase the affinity, the binding domain of the IL-8 bound to the small peptide was modeled and a Zn(II) coordination site at the interface of the IL-8/peptide complex was created by introducing a His residue in the peptide (FIG. 5).

It is not easy to find candidate sites within a given protein where the geometry of the polypeptide chain matches that of metal-binding sites in known protein structures. However, by choosing a site in the peptide that is inherently flexible, it is possible to engineer a Zn binding site. The flexibility of the site will allow adjusting the binding of Zn in its preferred geometry. This approach has been successfully applied to design an adaptable metal-binding site in T4 lysozyme (Wray et al., 2000). Using these criteria one may introduce His into the most flexible regions of the peptides derived from the N-terminal domain of CXCR1. Based on the NMR structure of the peptide bound to IL-8, residues 7-12 are the most flexible.

This newly created coordination site for Zn(II) at the interface of the IL-8/peptide was similar to naturally occurring Zn(II) coordination sites visualized in several crystal structures of soluble proteins, in which Zn(II) plays a key role in protein stability and enhances the affinity of protein-protein interactions. For instance, the Zn(II)-mediated interaction of human growth hormone (hGH) with the extracellular domain of the prolactin receptor (hPRLbp) is a naturally occurring example of such an interaction (Matthews and Wells, 1994). In the absence of Zn(II), hGH binds hPRLbp with relatively low affinity (Kd=270 nM). In the presence of Zn(II), however, the binding affinity increased about 8,000-fold (Kd=33 pM).

As shown in FIG. 6, inhibition of IL-8 binding to neutrophil membranes by the His-peptide was 125-fold better (Ki=1.12 μM) than in the absence of Zn(II) (Ki=141 μM). In contrast, the effect of the wild-type N-terminal CXCR1 peptide on the binding reaction was the same in the presence and absence of Zn(II). This finding strongly suggests that the binding of Zn(II) to the newly created metal site at the interface of the His-peptide of CXCR1-IL-8 complex has increased the binding affinity of the peptide by more than 100-folds. Accordingly, one may create a very high affinity neutralizing peptide to IL-8 by optimizing the Zn(II) coordination site using His-scanning substitutions on the N-terminal peptide of CXCR1.

TABLE 1
Anti-IL-8 Peptides

| | |
|---|---|
| acTWFEDEFANATGMPPVEKDYSP | (SEQ ID NO: 1) |
| acTWFEDEHANATGMPPVEKDYSP | (SEQ ID NO: 2) |
| acTWFEDEFHNATGMPPVEKDYSP | (SEQ ID NO: 3) |
| acTWFEDEFAHATGMPPVEKDYSP | (SEQ ID NO: 4) |
| acTWFEDEFANHTGMPPVEKDYSP | (SEQ ID NO: 5) |
| acTWFEDEFANAHGMPPVEKDYSP | (SEQ ID NO: 6) |
| acTWFEDEFANATHMPPVEKDYSP | (SEQ ID NO: 7) |
| acTWFEDEFANATGHPPVEKDYSP | (SEQ ID NO: 8) |
| acTWFEDEFANATGMPPVEKDYSP | (SEQ ID NO: 9) |

### EXAMPLE 2

Synthesis of High-Affinity Anti-Il-8 Peptides

The peptides shown in TABLE 1 are synthesized using a Milligen 9050 peptide synthesizer under continuous flow conditions employing Fmoc chemistry on PepsynK resin with AM linker. They were synthesized as N-terminal acetyl and c-terminal amides to prevent unwanted charged interactions at the termini. Resin cleavage conditions were TFA:phenol:EDT:anisole (95:2.5:2.5:2.5) for 2 h at room temperature. The resin was filtered and the solvent removed. The peptide was precipitated in ether, filtered and dried. Purification was by RP-HPLC on an Aquapore octyl column (20 micron, 100 mm×10 mm). The elution gradient consists of 95% A-95% B over 15 min where A=0.1% TFA in water and B=0.085% TFA in 70% acetonitrile. The peptides purity was tested by analytical RP-HPLC, and the structures was confirmed by mass spectroscopy, ¹H NMR and amino acid analysis.

The Ki of the receptor peptides was determined in an IL-8 ligand binding assay. Membranes of COS-1 cells transfected with the cDNA encoding CXCR1 were incubated in the presence of PBS, 0.1% BSA, 0.25 nM ¹²⁵I-labeled IL-8 and the His-peptide. Free and bound IL-8 was separated by rapid filtration as described previously (Thomas et al., 1991). A computerized curve-fit program (EBDA) was used to calculate Ki values for each His-peptide.

### EXAMPLE 3

Effects of Anti-Il-8 Peptides On Il-8-Induced Chemotaxis, Degranulation and Superoxide Production In Neutrophils

IL-8 secreted in response to inflammatory stimuli attracts neutrophils from the blood to the site of injury or infection by a process termed chemotaxis. IL-8 also induces exocytosis, leading to the release of enzymes from the granules and other storage organelles at the site of the inflammatory stimulus. Furthermore, IL-8 also triggers respiratory burst with the generation of superoxide, which is the precursor of microbicidal oxidants including hydrogen peroxide and hypochlorous acid. Modulation of the neutrophil response would be therapeutically desirable. Inhibition of these responses could prevent inflammation and tissue damage induced by products of activated neutrophils. The efficiency of the His-peptides derived from the N-terminal fragment of CXCR1 on IL-8-induced chemotaxis, degranulation and superoxide production by neutrophils is shown below.

To demonstrate the effect of His-peptides on IL-8-induced neutrophil responses, a cell line HL-60, which can differentiate into neutrophils by culturing the cells in DMSO, was used. Differentiated HL-60 cells were washed twice with pyrogen-free phosphate buffered saline (PBS) and then resuspended in 5.0 ml RPMI-1640 without phenol red (BioWhittaker, Walkersville, Md.) containing 10% heat-treated fetal calf serum (RPMI-FCS; HyClone, Logan, Utah). Calcein AM (Molecular Probes, Eugene, Oreg.) (5 μg/ml) were added to the 5.0 ml suspension of cells in RPMI-FCS and the cells were incubated for 30 minutes at 37° C. Differentiated HL-60 cells were washed twice with PBS, counted and resuspended in RPMI-FCS to the desired concentration.

The differentiated HL-60 cells were resuspended in cold (4° C.) “complete buffer” (PBS supplemented with 0.25% (w/v) bovine serum albumin, 0.1% (w/v) glucose, 0.9 mmol/l CaCl₂ and 0.5 mmol/l MgCl₂) and applied to the 48-well microchemotaxis chamber (Nuclepore). The chamber was assembled after thorough cleaning of all components with 0.1% NaOH and 0.03% sodium dodecylhydrogensulfate, followed by distilled water. The wells in the lower block were filled with 50 μl of the chemokines solutions. To assess random migration, 50 μl “complete buffer” was used in some lower wells. The polycarbonated (PC) membrane was then picked up by forceps and carefully placed on the lower block, a sealing gasket added on top of the membrane, and the upper part of the chamber tightened with screws. The upper-block wells was filled with cells in 50 μl “complete buffer”, and the chambers were incubated in humidified air with 5% CO₂ atmosphere at 37° C. for 15, 30, 35, 45 and 60 minutes. After incubation, the chamber was gently dismantled and the filter suspended between two clamps, the remaining cells on the upper surface of the membrane were gently wiped off using a windshield wiper blade. Cells that penetrated pores and attached on the lower surface were fixed in methanol for 10 seconds and stained with hematoxylin for 10 minutes. After rinsing with distilled water, the membrane was finally incubated in a bluing agent (2% MgSO₄ (w/v), 0.2% NaHCO₃ (w/v) in aqua dest.) for 5 minutes.

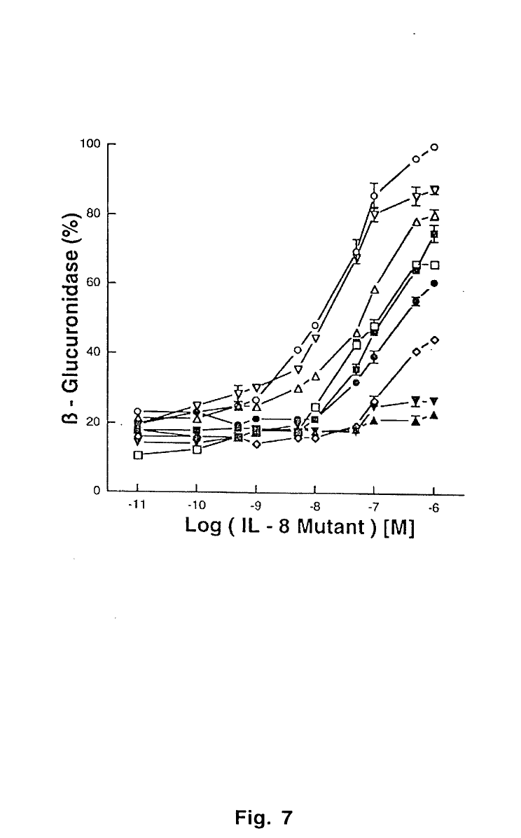

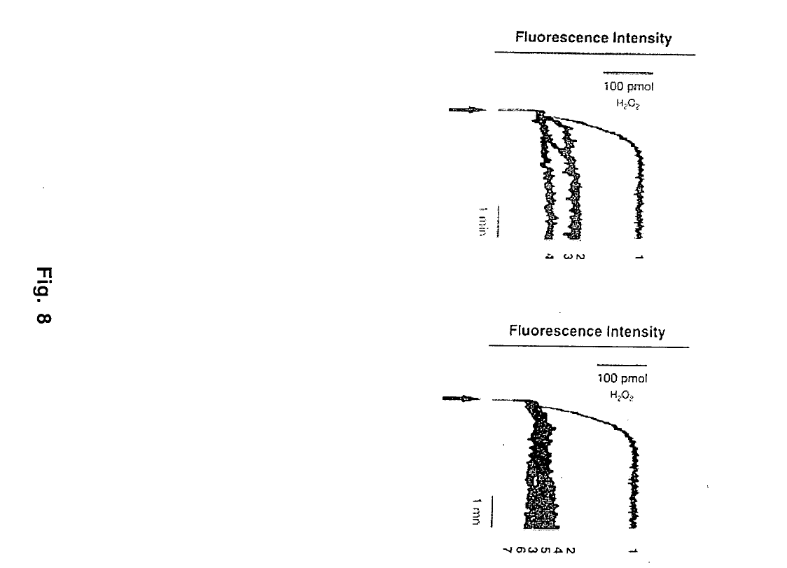

For β-glucuronidase assay, differentiated HL-60 cells (1×10⁷ cells/ml) were incubated in Hanks' balanced salt solution supplemented with 1% (w/v) bovine serum albumin, 2 mg/ml glucose, 4.2 mM NaHCO₃, 10 mM HEPES (pH 7.2) in the presence of IL-8 plus His-peptides. β-Glucuronidase assay was carried out according to the fluorometric assay described by Baly et al. (1997). Results from a typical assay with neutrophils and different IL-8 mutants are shown in FIG. 7.

Superoxide release is monitored by the continuous fluorometric measurement of 2,2′-dihydroxybiphenyl-5,5′-diacetate produced from p-hydroxyphenylacetate by the enzymatic reduction of H₂O₂ by horseradish peroxidase (Wymann et al., 1987). Differentiated HL-60 cells were suspended in physiological buffer containing 130 mM NaCl, 4.6 mM KCl, 1.1 mM KH₂PO₄, 1 mM CaCl₂, 5 mM glucose, and 20 mM HEPES (pH 7.4) at 1×10⁷ cells/ml. Cells were placed in physiological buffer containing 1 mM p-hydroxyphenylacetate, 20 units of horseradish peroxidase and 100 μM sodium azide in a continuously stirred cuvette maintained at 37° C. in a spectrofluorometer. Cells were stimulated with IL-8 in the presence of His-peptides and fluorescence intensity was measured using an excitation wavelength of 334 nm and an emission wavelength of 425 nm. Results from a typical assay with various IL-8 mutants are shown in FIG. 8.

### EXAMPLE 4

Conjugating Anti-Il-8 Peptides to Polymers to Enhance Plasma Half-Life and Stability

Short plasma half-life and poor stability are major problems of peptide-based drugs. A successful approach to overcome these limitations is to generate protein derivatives (Duncan, 2003). Conjugation of polyethylene glycol to peptides, PEGylation, is designed to increase both peptide solubility and stability. Most importantly, PEGylation can be used to prolong the plasma half-life of peptides by preventing their renal clearance and receptor-mediated protein uptake by the cells of the reticuloendothelial sytem. The clinical value of PEGylation is well established. For example, the PEG-adenosine deaminase was the first PEGylated protein to enter the market in 1990.

Anti-IL-8 peptides (2 mM) containing a C terminal lysine, separated by a glycine spacer, can be dissolved in 100 mM sodium phosphate buffer containing 1 mM of polyethylene glycol (PEG-NHS). This mixture is incubated for 24h at room temperature. This allows specific attachment of the NHS-derivatized PEG molecule to the lysine side chain. The PEGylated peptide can be purified by HPLC using a C4 column. Fractions containing the peptide-PEG conjugate can be lyophilized. The purity of the sample can be assessed by mass spectroscopy. The purified peptide-PEG conjugates can be tested for their ability to block IL-8 binding to its receptor as described above.

PEGylation of anti-IL-8 peptides is not likely to inactivate the peptides because it has been found that extension of the 21-amino acids peptides at the C- or N-terminus did not affect the blocking effect of the peptides. Nevertheless, the anti-IL-8 peptides can be PEGylated at the N-terminus to overcome potential problems of peptide inactivation. Moreover, the anti-IL-8 peptides can be PEGylated at both the C- and N-terminus to demonstrate that the stability of the peptides is enhanced without affecting peptide activities.

The following references were cited herein:

Baldwin et al. (1991). Crystal structure of interleukin 8: symbiosis of NMR and crystallography. Proc Natl Acad Sci USA 88:502-506.

Baly et al. (1997). Biological assays for C—X—C chemokines. Methods Enzymol 287:69-88.

Duncan (2003). The dawning era of polymer therapeutics. Nature Rev Drug Discovery 2:347-360.

Gayle et al. (1993). Importance of the amino terminus of the interleukin-8 receptor in ligand interactions. J Biol Chem 268:7283-7289.

Matthews and Wells. (1994). Engineering an interfacial zinc site to increase hormone-receptor affinity. Chem Biol 1:25-30.

Skelton et al. (1999). Structure of a CXC chemokine-receptor fragment in complex with interleukin-8. Structure Fold Des 7:157-168.

Suetomi et al. (1999). Differential mechanisms of recognition and activation of interleukin-8 receptor subtypes. J Biol Chem 274:11768-11772.

Thomas et al. (1991). The interleukin-8 receptor is encoded by a neutrophil-specific cDNA clone, F3R. J Biol Chem 266:14839-14841.

Wray et al. (2000). Use of a non-rigid region in T4 lysozyme to design an adaptable metal- binding site. Protein Eng 13:313-321.

Wymann et al. (1987). Chemiluminescence detection of H₂O₂ produced by human neutrophils during the respiratory burst.

Anal Biochem 165:371-378.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A chemokine peptide antagonist, said antagonist comprises the N-terminal domain of a CXCR1 receptor, wherein at least one native amino acid of said domain is replaced by one or more histidine residues, thereby forming Zn(II) binding site(s) in said domain that bind said chemokine in the presence of Zn(II).

2. The chemokine peptide antagonist of claim 1, wherein said N-terminal domain is at least 21 amino acids long.

3. The chemokine peptide antagonist of claim 1, wherein said CXCR1 receptor is human CXCR1 receptor or rabbit CXCR1 receptor.

4. The chemokine peptide antagonist of claim 1, wherein said chemokine is IL-8.

5. The chemokine peptide antagonist of claim 1, wherein said peptide antagonist has an amino acid sequence of SEQ ID NO:1.

6. The chemokine peptide antagonist of claim 1, wherein said peptide antagonist has an amino acid sequence of SEQ ID NO:2.

7. The chemokine peptide antagonist of claim 1, wherein said peptide antagonist has an amino acid sequence of SEQ ID NOs:3.

8. The chemokine peptide antagonist of claim 1, wherein said peptide antagonist has an amino acid sequence of SEQ ID NO:4.

9. The chemokine peptide antagonist of claim 1, wherein said peptide antagonist has an amino acid sequence of SEQ ID NO:5.

10. The chemokine peptide antagonist of claim 1, wherein said peptide antagonist has an amino acid sequence of SEQ ID NO:6.

11. The chemokine peptide antagonist of claim 1, wherein said peptide antagonist has an amino acid sequence of SEQ ID NO:7.

12. The chemokine peptide antagonist of claim 1, wherein said peptide antagonist has an amino acid sequence of SEQ ID NO:8.

13. The chemokine peptide antagonist of claim 1, wherein said peptide antagonist has an amino acid sequence of SEQ ID NO:9.

14. A pharmaceutical composition comprising the chemokine peptide antagonist, said antagonist comprises the N-terminal domain of a CXCR1 receptor, wherein at least one native amino acid of said domain is replaced by one or more histidine residues, thereby forming Zn(II) binding site(s) in said domain that bind said chemokine in the presence of Zn(II);
and a pharmaceutical acceptable carrier.

15. The pharmaceutical composition of claim 14, wherein said N-terminal domain is at least 21 amino acids long.

16. The pharmaceutical composition of claim 14, wherein said CXCR1 receptor is human CXCR1 receptor or rabbit CXCR1 receptor.

17. The pharmaceutical composition of claim 14, wherein said chemokine is IL-8.

18. The pharmaceutical composition of claim 14, wherein said peptide antagonist has an amino acid sequence of SEQ ID NO:1.

19. The pharmaceutical composition of claim 14, wherein said peptide antagonist has an amino acid sequence of SEQ ID NO:2.

20. The pharmaceutical composition of claim 14, wherein said peptide antagonist has an amino acid sequence of SEQ ID NOs:3.

21. The pharmaceutical composition of claim 14, wherein said peptide antagonist has an amino acid sequence of SEQ ID NO:4.

22. The pharmaceutical composition of claim 14, wherein said peptide antagonist has an amino acid sequence of SEQ ID NO:5.

23. The pharmaceutical composition of claim 14, wherein said peptide antagonist has an amino acid sequence of SEQ ID NO:6.

24. The pharmaceutical composition of claim 14, wherein said peptide antagonist has an amino acid sequence of SEQ ID NO:7.

25. The pharmaceutical composition of claim 14, wherein said peptide antagonist has an amino acid sequence of SEQ ID NO:8.

26. The pharmaceutical composition of claim 14, wherein said peptide antagonist has an amino acid sequence of SEQ ID NO:9.

27. A method of treating an individual having or at risk of having a disorder or disease mediated in part by a chemokine, said method comprises the step of administering a pharmacologically effective amount of the pharmaceutical composition of claim 14 to said individual.

28. A method of treating an individual having or at risk of having a disorder or disease mediated in part by a chemokine, said method comprises the step of administering a pharmacologically effective amount of the pharmaceutical composition of claim 18 to said individual.

29. A method of treating an individual having or at risk of having a disorder or disease mediated in part by a chemokine, said method comprises the step of administering a pharmacologically effective amount of the pharmaceutical composition of claim 19 to said individual.

30. A method of treating an individual having or at risk of having a disorder or disease mediated in part by a chemokine, said method comprises the step of administering a pharmacologically effective amount of the pharmaceutical composition of claim 20 to said individual.

31. A method of treating an individual having or at risk of having a disorder or disease mediated in part by a chemokine, said method comprises the step of administering a pharmacologically effective amount of the pharmaceutical composition of claim 21 to said individual.

32. A method of treating an individual having or at risk of having a disorder or disease mediated in part by a chemokine, said method comprises the step of administering a pharmacologically effective amount of the pharmaceutical composition of claim 22 to said individual.

33. A method of treating an individual having or at risk of having a disorder or disease mediated in part by a chemokine, said method comprises the step of administering a pharmacologically effective amount of the pharmaceutical composition of claim 23 to said individual.

34. A method of treating an individual having or at risk of having a disorder or disease mediated in part by a chemokine, said method comprises the step of administering a pharmacologically effective amount of the pharmaceutical composition of claim 24 to said individual.

35. A method of treating an individual having or at risk of having a disorder or disease mediated in part by a chemokine, said method comprises the step of administering a pharmacologically effective amount of the pharmaceutical composition of claim 25 to said individual.

36. A method of treating an individual having or at risk of having a disorder or disease mediated in part by a chemokine, said method comprises the step of administering a pharmacologically effective amount of the pharmaceutical composition of claim 26 to said individual.

37. The method of claim 27, wherein said pharmaceutical composition is delivered across the skin or administered systemically.

38. The method of claim 27, wherein said chemokine is IL-8.

39. The method of claim 27, wherein said pharmaceutical composition comprises chemokine peptide antagonist having an amino acid sequence selected from the group consisting of SEQ ID NOs:1-9.

40. The method of claim 27, wherein said disorder or disease is selected from the group consisting of as ischemia-reperfusion injury, psoriasis, microbial infections, HIV-1 infection, allergies, arthritis, arteriosclerosis, chronic inflammation, acute inflammation, gout, acute pseudogout, acute gouty arthritis, rheumatoid arthritis, osteoarthritis, allograft rejection, chronic transplant rejection, asthma, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, septic shock, endotoxic shock, urosepsis, glomerulonephritis, lupus nephritis, thrombosis, graft vs. host reaction, angiogenesis, NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, is kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies, and solid tumors arising from lymphomas.
